(19) United States
(12) Patent Application Publication
(10) Pub. No.: US 2002/0068038 A1
(43) Pub. Date: Jun. 6, 2002

(54) Foaming tablet for cleaning the oral cavity and preparation method thereof

(76) Inventor: Joo Hwan Yang, Bucheon (KR)

Correspondence Address: JACOBSON HOLMAN PLLC, 400 SEVENTH STREET N.W., SUITE 600, WASHINGTON, DC, 20004, US

(21) Appl. No.: 09/985,589

(22) Filed: Nov. 5, 2001

Related U.S. Application Data
(63) Continuation-in-part of application No. 09/600,864, filed on Jul. 27, 2000.
Application No. 09/600,864 is a 371 of international application No. PCT/KR99/00732, filed on Dec. 3, 1999.

(30) Foreign Application Priority Data
Dec. 5, 1998 (KR) 1998-53215

(51) Int. Cl. A61L 9/04; A61K 7/18

(52) U.S. Cl. 424/44; 424/52

(57) ABSTRACT

The present invention relates to a foaming tablet for cleaning the oral cavity, more particularity, a foaming tablet prepared by mixing and tabletting the oral cavity cleaning components and foaming agent to be used for the efficient cleaning agent of oral cavity.

This is continuation-in-part application of U.S. Ser. No. 09,600,864.

### TECHNICAL FIELD

The present invention relates to a foaming tablet for cleaning the oral cavity, more particularity, a foaming tablet prepared by mixing and tabletting the oral cavity cleaning components and foaming agent to be used for the efficient cleaning agent of oral cavity.

In detail, the foaming tablet comprises i) a microcrystalline cellulose as the tabletting basic material ; ii) a foaming agent selected from the group consisting of sodium bicarbonate, ammonium bicarbonate, potassium bicarbonate, iron carbonate and mixture of them ; iii) a grinding agent ; iv) a xylitol ; v) an organic acid ; vi) a tooth protecting agent ; and vii) other additives for taste, flavor and color. The foaming tablet of the present invention provides the convenience of use and storage.

### BACKGROUND ART

In case of the toothpaste, it requires additional device and time to clean the oral cavity, even though the complete grinding and removal of tartar can be accomplished by the movement of toothbrush. On the other hand, the liquid type of oral cavity cleaner requires additional device, such as, cup or other pot due to the large volume and weight.

To solve above problems, a tablet type of oral cavity cleaner has been developed in the present invention, which has a lot of merits, for example, i) convenience of storage, ii) not requiring the additional device, and iii) cleaning tooth and oral cavity with good taste and flavor.

The commercially marketed cleaning agents for oral cavity can be examplified as follows, i) drink tea type liquid agent, ii) candy type solid agent, and iii) liquid or gel type toothpaste. Followings are gel type toothpaste developed up to now.

EP 0 303 520 A disclosed the toothpaste comprising i) agent for removing tartar containing the methaphosphate soda, anhydro calcium phosphite, and ii) protecting agent for tooth color change containing calcium peroxide, calcium perborate. U.S. Pat. No. 5,122,365 disclosed the toothpaste comprising hydrogen peroxide, sodium benzoate and titanium oxide for protecting color change. Further, U.S. Pat. No. 4,986,981 disclosed the toothpaste comprising citric acid, papain and aluminum oxide.

EP 0 279 130 A disclosed the powder type toothpaste comprising hydrogen peroxide and sodium bicarbonate to improve the cleaning effect of tooth. However, such toothpaste has handicap to induce the damage of oral cavity. Korean Laid Open Patent No. 96-00198 disclosed the toothpaste having whitening effect of tooth using zeolite, protease and peroxide. Also, Korean Laid Open Patent No. 95-28755 disclosed the toothpaste having the effect of plague removal and tooth protection using sodium bicarbonate particle. Korean Laid Open Patent No. 97-25597 disclosed the liquid type toothpaste by mixing bamboo salt and catechin to remove the oral dusty odor. Further, Korean Laid Open Patent No. 98-00424 and 96-20996 disclosed the toothpaste having tooth protection effect in addition of salt, Lactococcus sp. HY49 or bacteriocin.

As liquid type cleaner, a lot of oral cavity cleaning agents have been developed, for example, using CPC and xylitol for protecting tooth. On the other hand, trade names, Score (Mindspring Co.), Oral rinse (Zinnelle Co.) and Retradex (Rowpar pharm. Inc.) have been developed to removing the plague and to protect tooth disease using ClO₂, aloevera or chlorophyll. Further, the pouch pack has been used as package of liquid type tooth cleaner for cleaning oral cavity (SIP rinse/Profresh Co.).

U.S. Pat. No. 5,094,842 disclosed the toothpaste comprising tin compound, copper compound and vitamin C. Further, U.S. Pat. No. 4,563,293 disclosed the toothpaste comprising monoalkyl or dialkylether of dianhydroxitol. U.S. Pat. No. 4,335,102 disclosed the toothpaste comprising pitinic acid compound or tin compound as oral cavity cleaner, and calcium fluoride or soluble fluoride compound. Further, the long release type of toothpaste had been developed to spray the fluoride compound in oral cavity (U.S. Pat. No. 5,137,449 ; 5,049,0774 ; 5,137,449).

On the other hand, U.S. Pat. No. 4,417,993 disclosed the cleaning tablet for artificial tooth comprising calcium carbonate and magnesium hydroxycarbonate. U.S. Pat. No. 3,888,976 disclosed the cleaning tablet comprising copper component for removing plague and zinc or strontium component for removing allergy. U.S. Pat. No. 5,670,138 disclosed the cleaning tablet comprising N-vinylpyrrolidone and acrylic acid to improve the grinding effect and flavor. Also, U.S. Pat. No. 4,243,655 disclosed the cleaning tablet comprising biotin to resist plague and acid formation. U.S. Pat. No. 5,529,788 disclosed the cleaning tablet comprising enzyme, surfactant and foaming agent.

Such cleaning tablet has many merits compared to toothpaste. Toothpaste requires additional time and device inspite of accomplishing complete grinding and removal of plague. However, the cleaning tablet is convenient for use and storage without requiring time and device, which can be used by chewing and washing out.

### DISCLOSURE OF INVENTION

The object of the present invention is to provide a foaming tablet for cleaning oral cavity comprising i) 30˜60 wt % of microcrystalline cellulose as the tabletting basic material ; ii) 20˜40 wt % of the foaming agent selected from the group consisting of sodium bicarbonate, ammonium bicarbonate, potassium bicarbonate, iron carbonate and mixture of them ; iii) 5˜20 wt % of the grinding agent selected from the group consisting of silica, precipitated silica, hydrated silica, silica gel, zirconium silicate, aluminosilicate and mixture of them ; iv) 2˜8 wt % of xylitol ; v) 3˜20 wt % of the organic acid ; vi) 0.05˜1.0 wt % of the tooth protecting agent ; and vii) other additives for taste, flavor and color.

The present invention also provides a foaming tablet, wherein said organic acid is selected from the group consisting of citric acid, tartaric acid, malic acid, gluconic acid, ascorbic acid, succinic acid, propionic acid and mixture of them ; and said tooth protecting agent is selected from the group consisting of sodium fluoride, thymol and other fluoride compound.

Also, the foaming tablet of the present invention further comprises anion and/or nonion surfactant as auxiliary foaming agent selected from the group consisting of sodium laurylsulfate, sodium N-lauroylsalcosylate, N-acyl glutamate, saccharose fatty ester, polyoxyethylene hardened castor oil, sorbitan fatty ester, polyoxyethylene polyoxypropylene copolymer and mixture of them.

The other object of the present invention is to provide a foaming tablet having the convenience of use and storage with sufficient oral cavity cleaning effect.

### BRIEF DESCRIPTION OF DRAWINGS

FIG. 1 is a schematic diagram for determining the minimum concentration of disinfection activity against dental bacteria.

FIG. 2 is a diagram for determining the minimum disinfection concentration against dental bacteria by cultivating the lowest concentration test material in LRBB agar medium.

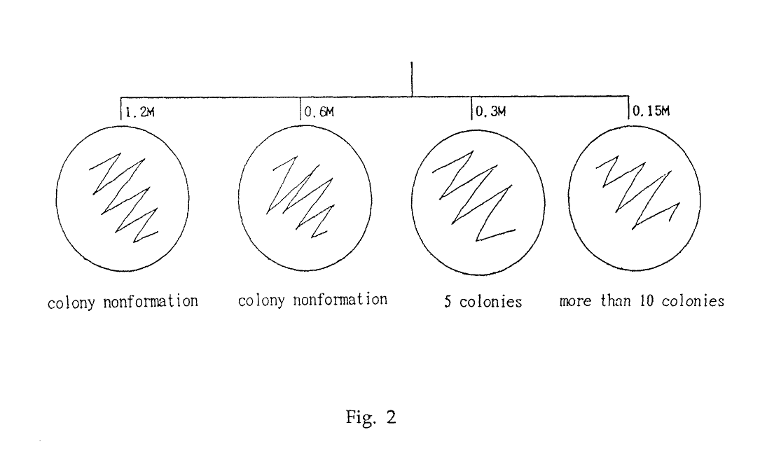

The lowest concentration shows nonopaque of test tube having 1.0M of salt and MH medium.

The minimum disinfection concentration is determined when less than 10 colonies are grown in LRBB agar plate medium.

FIG. 3 is a graph which shows the disinfection effect according to the concentration of sodium bicarbonate.

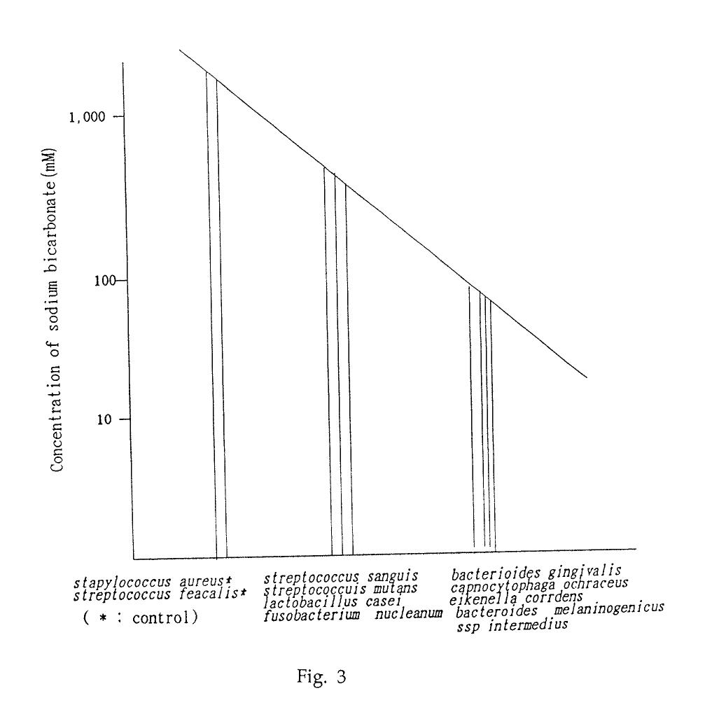

### BEST MODE FOR CARRYING OUT THE INVENTION

The tabletting basic material of the present invention shall be microcrystalline cellulose. The reason why the microcrystalline cellulose is required is as follows.

Microcrystalline cellulose is a polysaccharide having β-1,4 bond between glucoses of M.W. 25,000˜50,000 generally used as base material of food and drug because of its safety. It also has a property to adsorb or maintain the volatile tasting or flavoring agent. Further, it can be used as adsorbent, binder, diluent and disintegrant.

Therefore, microcrystalline cellulose has to be used as basic material of foaming tablet, because it can adsorb or maintain volatile tasting or flavoring agent and fluoride compound for cleaning cavity and removing the mouth smell. Further, it also has the function as binding agent which gives tissue feeling.

The foaming agent selected from the group consisting of sodium bicarbonate, ammonium bicarbonate, potassium bicarbonate, iron carbonate and mixture of them can be used not only as foaming agent, but also as oral cavity cleaning agent. Further, sodium bicarbonate has been used as baking powder due to its safety, which is water soluble white powder and releases carbon dioxide at the time of solving in water. On the other hand, sodium bicarbonate has also the tooth protecting effect by neutralizing the organic acid raised by plague as well as the suppression effect against oral cavity bacteria.

As grinding agent, it can be selected from the group consisting of silica, precipitated silica, hydrated silica, silica gel, zirconium silicate, aluminosilicate and mixture of them. Further, silica is preferred, especially, Tixosil-73K.

Xylitol is 5 carbon sugar alcohol, has anticariogenic property which does not induce acid formation, and has a sweetness equal to sucrose. Also, xylitol has substantively lower viscosity and negative heat effect when dissolved in the solution. Further, xylitol has the suppression effect against oral cavity bacteria including pneumococcus. Therefore, the addition of xylitol in foaming tablet is required to avoid the growth of bacteria in oral cavity as well as to enhance the sweetness in the tablet.

As an organic acid, it can be selected from the group consisting of citric acid, tartaric acid, malic acid, gluconic acid, ascorbic acid, succinic acid, propionic acid and mixture of them.

Anion and/or nonion surfactant can be used as auxiliary foaming agent, which is selected from the group consisting of sodium laurylsulfate, sodium N-lauroylsalcosylate, N-acyl glutamate, saccharose fatty ester, polyoxyethylene hardened castor oil, sorbitan fatty ester, polyoxyethylene polyoxypropylene copolymer and mixture of them.

Sodium fluoride and other fluoride compound have the effect as tooth protecting agent, which also gives strengthening effect to the dental tissue. Further, it provides the effect for removing the tartar.

Thymol is also used as tooth protecting agent, because it can have a property to suppress the growth of bacteria in oral cavity.

As flavoring agent, it can be selected from the group consisting of orange flavor, strawberry flavor, grape flavor, peppermint oil, spearmint oil, menthol, carvone, anerol, oigenol, wintergreen, sage, eucalypto oil, methyl salicylate, fruit extract and mixture of them.

As sweetening agent, it can be selected from the group consisting of lactose, maltose, lactitol, saccharin sodium, aspartam, stevioside, persimmon flavor and mixture of them.

As tasting agent, it can be selected from the group consisting of sage, rosemary, green tea, persimmon extract, shanpinion extract and mixture of them.

As an preferred embodiment, the mixture of sodium laurylsulfate, rosemary, green tea powder, shanpinion extract and persimmon extract has the improved function for removing the unnecessary material from the oral cavity quickly by the fast dispersion and penetration in the oral cavity. The mixture of citric acid, tartaric acid, malic acid, gluconic acid, ascorbic acid, succinic acid and propionic acid has good flavor and foaming activity. In this composition, the aspartam has a role of sweetener, and menthol has a role of flavoring agent.

Decayed tooth is induced by the bacteria to the person whose enamel of tooth is not sufficiently strong, eating a lot of sugar contents. In such decayed tooth, the tartar will grow to be bleeding or inflammation of teethridge. In this case, mouth smell will occur generally. Such a mouth smell has a bad influence to the social life of the person. Therefore, the cleaning of oral cavity is very important to have a good relation to other people. The oral cavity cleaner developed so far is mainly a liquid type. The solid or powder type of oral cavity cleaner is very rare.

Therefore, the foaming tablet for cleaning oral cavity of the present invention provides a lot of following merits ; i) good tissue feeling when chewing it ; ii) quick generation of foam by the good dissolution in oral cavity ; iii) excellent tooth protecting function ; and iv) good flavor and cleaning effect.

The present invention will be more specifically explained by the following examples. However, it should be understood that the examples are intended to illustrate but not in any manner to limit the scope of the present invention.

### EXAMPLES 1˜12

### Preparation method

In order to prepare foaming composition, each components are mixed by following steps.

1) Pre-mixing Step

Coloring agent, the natural pigment of TS melon, is added to the distilled water to make 3.5% solution. Such coloring solution is adsorbed to 30˜60 wt % of microcrystalline cellulose. Then, the mixture is dried for 2˜3 hours at 65° C. Obtained material is crashed through 40 mesh sieve.

2) Mixing Step

To the pre-prepared material in above step, following components; i) 20˜40 wt % of the foaming agent selected from the group consisting of sodium bicarbonate, ammonium bicarbonate, potassium bicarbonate, iron carbonate and mixture of them ; ii) 5˜20 wt % of the grinding agent, such as, silica (Tixosil-73K) ; iii) 2˜8 wt % of xylitol ; iv) 2˜20 wt % of the organic acid selected from the group consisting of citric acid, tartaric acid, malic acid, gluconic acid, ascorbic acid, succinic acid, propionic acid and mixture of them ; v) 0.1˜0.5 wt % of menthol ; vi) 0.1˜0.5 wt % of sodium fluoride and thymol ; vii) some amount of peppermint flavor (or spearmint, orange, strawberry, fruity flavor) ; viii) some amount of sodium laurylsulfate ; and ix) some amount of rosemary, green tea powder, persimmon extract, shanpinion extract and mixture of them are mixed as shown in Table 1.

3) Tabletting Step

The mixture in above step is tabletted using the suitable device.

TABLE 1

| | Mixing ratio (%) | | | | | | | | | | | |
|---|---|---|---|---|---|---|---|---|---|---|---|---|
| Component | Exp. 1 | Exp. 2 | Exp. 3 | Exp. 4 | Exp. 5 | Exp. 6 | Exp. 7 | Exp. 8 | Exp. 9 | Exp. 10 | Exp. 11 | Exp. 12 |
| microcrystalline | 48.04 | 59.44 | 53.62 | 57.44 | 46.24 | 34.15 | 45.86 | 43.30 | 38.49 | 31.54 | 45.40 | 51.59 |
| cellulose | | | | | | | | | | | | |
| sodium | 26.90 | 20.40 | 27.96 | 23.00 | 30.65 | 30.65 | 30.65 | 29.23 | 32.69 | 37.38 | 38.20 | 26.96 |
| bicarbonate, | | | | | | | | | | | | |
| ammonium | | | | | | | | | | | | |
| bicarbonate, | | | | | | | | | | | | |
| potassium | | | | | | | | | | | | |
| bicarbonate, iron | | | | | | | | | | | | |
| carbonate or | | | | | | | | | | | | |
| mixture of them | | | | | | | | | | | | |
| Tixosil 73K | 7.65 | 8.84 | 5.35 | 10.42 | 11.95 | 12.65 | 11.95 | 14.62 | 15.88 | 10.35 | 6.18 | 10.66 |
| citric acid, tartaric | 13.0 | 6.85 | 8.74 | 4.75 | 6.74 | 18.15 | 6.74 | 7.35 | 8.34 | 9.68 | 5.52 | 5.74 |
| acid, malic acid, | | | | | | | | | | | | |
| gluconic acid, | | | | | | | | | | | | |
| ascorbic acid, | | | | | | | | | | | | |
| succinic acid, | | | | | | | | | | | | |
| propionic acid or | | | | | | | | | | | | |
| mixture of them | | | | | | | | | | | | |
| xylitol | 3.00 | 3.00 | 3.00 | 3.00 | 3.00 | 3.00 | 3.00 | 3.00 | 3.00 | 3.00 | 3.00 | 3.00 |
| menthol | 0.10 | 0.20 | 0.25 | 0.30 | 0.30 | 0.30 | 0.20 | 0.40 | 0.50 | 0.30 | 0.30 | 0.30 |
| peppermint | 0.30 | 0.40 | 0.50 | 0.50 | 0.50 | — | — | 0.50 | — | 0.50 | 0.50 | 0.50 |
| sodium fluoride | 0.10 | 0.15 | 0.15 | 0.15 | 0.15 | 0.15 | 0.15 | 0.20 | 0.15 | 0.30 | 0.15 | 0.50 |
| natural pigment | 0.40 | 0.40 | 0.40 | 0.40 | 0.40 | 0.40 | 0.40 | 0.40 | 0.40 | 0.40 | 0.40 | 0.40 |
| of TS melon | | | | | | | | | | | | |
| sodium lauryl- | 0.01 | 0.02 | 0.03 | 0.04 | 0.05 | 0.05 | 0.05 | 0.05 | 0.05 | 0.05 | 0.05 | 0.05 |
| sulfate | | | | | | | | | | | | |
| green tea powder | — | 0.30 | — | — | — | — | — | — | — | — | — | — |
| fruity flavor | 0.5 | — | — | — | — | — | — | 0.50 | — | — | — | — |
| orange flavor | — | — | — | — | — | 0.50 | — | — | — | — | — | — |
| strawberry flavor | — | — | — | — | — | — | — | — | 0.50 | — | — | — |
| spearmint flavor | — | — | — | — | — | — | 1.00 | — | — | — | — | 0.3 |
| sodium | — | — | — | — | — | — | — | 0.15 | — | — | — | — |
| fluorophosphate | | | | | | | | | | | | |
| rosemary | — | — | — | — | — | — | — | 0.30 | — | — | — | — |
| persimmon | — | — | — | — | — | — | — | — | — | 6.50 | — | — |
| extract | | | | | | | | | | | | |
| shanpinion extract | — | — | — | — | — | — | — | — | — | — | 0.30 | — |
| Total | 100.00 | 100.00 | 100.00 | 100.00 | 100.00 | 100.00 | 100.00 | 100.00 | 100.00 | 100.00 | 100.00 | 100.00 |

### EXAMPLES 13

### Disinfection Effect Against Dental Bacteria and Dental Cavity Cleaning Effect Of the Present Foaming Tablet Compared To Other Marketed Products

1) Introduction

To determine the minimum disinfection effect against dental bacteria, each component having disinfection effect which are sodium bicarbonate, sodium chloride, ammonium bicarbonate, potassium bicarbonate, magnesium sulfate, sodium laurylsulfate and mixture of sodium bicarbonate and sodium laurylsulfate is experimented. The result will show the disinfection function of the foaming tablet in the present invention compared to other marketed products.

2) Method

i) Selection and Storage Of Dental Bacteria 11 dental bacteria selected and stored are as following bacteria, Streptococcus sanguis, Streptococcuis mutans, Lactobacillus casei, Acetinobacillus, Bacterioides gingivalis, Capnocytophaga ochraceus, Eikenella corrdens, Fusobacterium nucleanum, Bacteroides melaninogenicus ssp intermedius, Stapylococcus aureus and

Streptococcus feacalis. They are stored in laked-rabbit brucella(LRBB) agar medium in anaerobic condition (85% N₂, 10% H₂, 5% CO₂).

ii) Reagent and Material

Sodium bicarbonate, sodium chloride, ammonium bicarbonate, potassium bicarbonate, magnesium sulfate, sodium laurylsulfate and mixture of sodium bicarbonate and sodium laurylsulfate are used as reagent. Further, commercially marketed toothpaste, liquid type of oral cleaner and foaming tablet of the present invention are used as material.

iii) Test for determining disinfection concentration

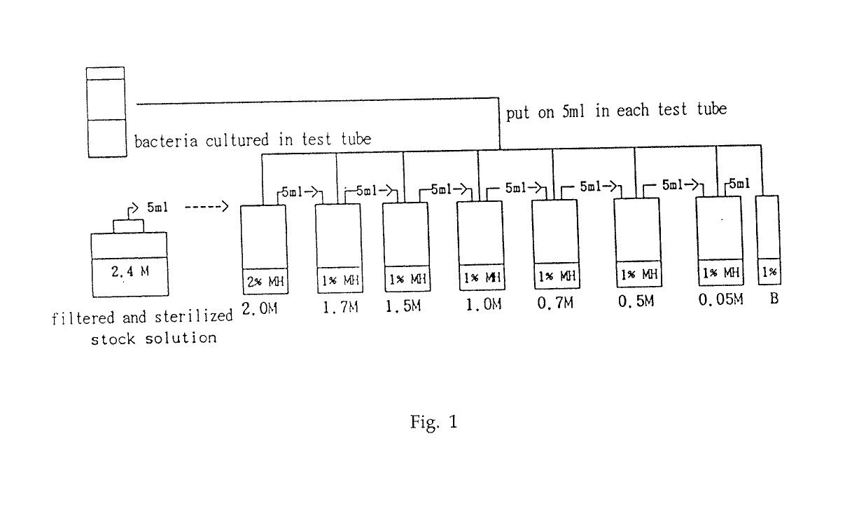

To determine disinfection concentration against dental bacteria, sodium bicarbonate, sodium chloride, ammonium bicarbonate, potassium bicarbonate, magnesium sulfate, sodium laurylsulfate and mixture of sodium bicarbonate and sodium laurylsulfate are diluted as shown in FIG. 1 using Mueller Hinton (MH) medium. Stock solution (1.2˜4.8M of salt solution) is filtered by 0.2 μm membrane, and Blank (B) is made without addition of salt. The strains prepared by 24 hours cultivation and 72 hours cultivation are mixed. Then, 0.1 ml of the solution contains 10⁵˜10⁷ bacteria.

After 96 hours anaerobic cultivation at 37° C., the lowest concentration of nonopaque test tube is determined as minimum disinfection concentration. To confirm the minimum disinfection concentration, the strain is transferred and cultivated in LRBB agar plate medium. Then, minimum disinfection concentration of the strain is determined by detecting the number of colonies less than 10.

The cultivation of strain has been carried out in MH medium according to the standard method of #1 Macfarland. Further, the colony formation has been arried out in LRBB agar medium by 72˜96 hours' anaerobic cultivation. Each suspension of tested bacteria (10 μl) is put on shaedler agar (Difco) medium with or without salt dilution. In the plate medium, the cultivation is carried out at 37° C. for 5˜7 days.

iv) Disinfection Activity Of Bacteria

Various strains of bacteria are suspended in MH medium with 1M of sodium bicarbonate and maintain them at 37° C. in water bath. Such strains are diluted in LRBB agar medium and cultivate in plate medium to form colonies for 5˜7 days in anaerobic condition.

v) Comparison of oral cavity cleaning effect among present tablet, toothpaste and liquid type oral cleaner.

Preparation

The tablet prepared in this invention (700 mg) is dissolved and diluted in 19.7 g of phosphate buffer. The marketed toothpaste (1 g) is also dissolved and diluted in 19 g of phosphate buffer. The liquid type oral cleaner (20 g) is prepared. All 3 samples are sterilized in high pressure and 4 dental bacteria [ Streptococcus sanguis, Streptococcuis mutans, Lactobacillus casei and Acetinobacillus ] are prepared and diluted in the concentration of 10⁵˜10⁷.

Measuring the Number Of Dental Bacteria

20 ml of standard method agar medium is put on sterilized Petri dish at 45° C. 5 g of tested sample and 1 g of bacteria sample are mixed, stirred and cultivated. They are laid and solidified by laying in room temperature. The number of colony is measured after 7 days cultivation at 20° C. to calculate the number of dental bacteria.

3) Result

Table 2 shows the disinfection concentration of various salts to dental bacteria. The mixture of sodium laurylsulfate (SLS) and sodum bicarbonate shows improved disinfection activity compared to other salts. Further, sodium bicarbonate shows better effect of foam formation and safety. Of course, the disinfection activity of sodium bicarbonate is considered as the anion of bicarbonate.

TABLE 2

| Dental bacteria | NaHCO₃ | NaCl | KHCO₃ | NH₄CO₃ | MgSO₄ | NaHCO₃ + SLS |
|---|---|---|---|---|---|---|
| Streptococcus sanguis | 500 | 2,200 | 300 | 600 | 1,600 | 150 |
| Streptococcuis mutans | 450 | 1,300 | 600 | 700 | 1,700 | 200 |
| Lactobacillus casei | 500 | 1,200 | 600 | 400 | 1,500 | 150 |
| Acetinobacillus | 250 | 250 | 300 | 300 | 300 | 100 |
| Bacterioides gingivalis | 500 | 650 | 100 | 600 | 600 | 150 |
| Capnocytophaga ochraceus | 200 | 350 | 150 | 350 | 400 | 100 |
| Eikenella corrdens | — | 400 | 300 | 100 | — | — |
| Fusobacterium nucleanum | 450 | 400 | — | 300 | 300 | 100 |
| Bacteroides melaninogenicus | 100 | 650 | 50 | 150 | 200 | 50 |
| ssp intermedius | | | | | | |

Table 3 shows the improved disinfection effect of the tablet prepared in this invention compared to marketed toothpaste and liquid type oral cleaner.

TABLE 3

| | Marketed | | Liquid type |
|---|---|---|---|
| Dental bacteria | toothpaste | Foaming tablet | oral cleaner |
| Streptococcuis mutans | 6.6 × 10^{∠} | 5.5 × 10^{∠} | 5.7 × 10^{∠} |
| Streptococcus sanguis | 5.9 × 10^{∠} | 4.9 × 10^{∠} | 6.7 × 10^{∠} |
| Lactobacillus casei | 6.3 × 10^{∠} | 5.5 × 10^{∠} | 8.5 × 10^{∠} |
| Acetinobacillus | 5.5 × 10 | 2.6 × 10 | 4.2 × 10 |

### EXAMPLES 14

### Sensuous experiment

1) Panel

Panels are selected from 5 men and 5 women in age 25˜30.

2) Material

i) Marketed toothpaste: 2.5 g of toothpaste is used and brushed by toothbrush with 15 times up and down brushing. Then, 10 times washing out is carried out using water.

ii) Foaming tablet of the present invention prepared in Exp. 4: 1 tablet is selected and chewing to clean tooth and tongue. Water is used for gargling.

iii) Liquid type oral cleaner: 20 ml of oral cleaner is put in the mouth and gargled 15 times in the mouth.

3) Method

The functions of following items are measured.

i) Removal of unnecessary thing in the tooth: The level of removing the unnecessary thing in the tooth is measured before and after test.

ii) Removal of mouth odor: The level of removing mouth odor is measured before and after test.

iii) Tissue feeling: The level of tissue feeling is measured when applied in the mouth.

iv) Taste and flavor: The level of taste and flavor is measured when applied in the mouth.

v) Convenience: The level of convenience is measured when applied in the mouth.

4) Result

The result is counted by the score tested by 10 panels, and the score is in each item given the number by following rule ;

the worst 1, 2, 3, 4, 5, 6, 7, 8, 9, 10 the best

Table 4 shows the result. The removal of unnecessary thing in the tooth is excellent when using toothpaste. However, other items, removal of mouth odor ; tissue feeling ; taste and flavor ; convenience are excellent when using foaming tablet of the present invention.

TABLE 4

| | Marketed toothpaste | | | | | Foaming tablet | | | | | Liquid type oral cleaner | | | | |
|---|---|---|---|---|---|---|---|---|---|---|---|---|---|---|---|
| Panel | i | ii | iii | iv | v | i | ii | iii | iv | v | i | ii | iii | iv | v |
| 1 | 8 | 6 | 3 | 3 | 5 | 6 | 8 | 10 | 8 | 10 | 4 | 8 | 3 | 8 | 4 |
| 2 | 7 | 7 | 3 | 4 | 4 | 7 | 7 | 9 | 10 | 10 | 5 | 7 | 3 | 8 | 3 |
| 3 | 8 | 5 | 4 | 3 | 5 | 5 | 6 | 9 | 9 | 10 | 4 | 6 | 3 | 8 | 4 |
| 4 | 9 | 6 | 3 | 4 | 4 | 6 | 7 | 9 | 9 | 10 | 3 | 7 | 4 | 7 | 5 |
| 5 | 7 | 5 | 6 | 5 | 6 | 6 | 8 | 8 | 8 | 10 | 3 | 8 | 5 | 8 | 3 |
| 6 | 8 | 7 | 3 | 5 | 7 | 6 | 8 | 10 | 7 | 9 | 3 | 7 | 4 | 8 | 3 |
| 7 | 7 | 8 | 5 | 4 | 3 | 7 | 9 | 10 | 9 | 10 | 3 | 6 | 5 | 9 | 3 |
| 8 | 9 | 5 | 4 | 5 | 3 | 5 | 8 | 9 | 9 | 9 | 2 | 6 | 4 | 7 | 2 |
| 9 | 7 | 6 | 3 | 6 | 4 | 5 | 9 | 10 | 10 | 9 | 4 | 5 | 5 | 8 | 4 |
| 10 | 8 | 7 | 2 | 7 | 3 | 6 | 9 | 9 | 9 | 10 | 3 | 5 | 5 | 7 | 5 |
| Total | 78 | 62 | 36 | 46 | 44 | 59 | 79 | 93 | 88 | 97 | 34 | 65 | 41 | 78 | 36 |

```
*i: Removal of unnecessary thing in the tooth
  ii: Removal of mouth odor
  iii: Tissue feeling
  iv: Taste and flavor
  v: Convenience
```

What is claimed is:

1. A foaming tablet for cleaning oral cavity comprising i) 30˜60 wt % of microcrystalline cellulose as the tabletting basic material ; ii) 20˜40 wt % of the foaming agent selected from the group consisting of sodium bicarbonate, ammonium bicarbonate, potassium bicarbonate, iron carbonate and mixture of them ; iii) 5˜20 wt % of the grinding agent selected from the group consisting of silica, precipitated silica, hydrated silica, silica gel, zirconium silicate, aluminosilicate and mixture of them ; iv) 2˜8 wt % of xylitol ; v) 3˜20 wt % of the organic acid ; vi) 0.05˜1.0 wt % of the tooth protecting agent ; and vii) other additives for taste, flavor and color.

2. The foaming tablet according to claim 1, wherein said organic acid is selected from the group consisting of citric acid, tartaric acid, malic acid, gluconic acid, ascorbic acid, succinic acid, propionic acid and mixture of them.

3. The foaming tablet according to claim 1, wherein said tooth protecting agent is selected from the group consisting of sodium fluoride, thymol and other fluoride compound.

4. The foaming tablet according to claims 1, further comprising anion and/or nonion surfactant as auxiliary foaming agent selected from the group consisting of sodium laurylsulfate, sodium N-lauroylsalcosylate, N-acyl glutamate, saccharose fatty ester, polyoxyethylene hardened castor oil, sorbitan fatty ester, polyoxyethylene polyoxypropylene copolymer and mixture of them.
